(19) United States
(12) Patent Application Publication
(10) Pub. No.: US 2015/0025357 A1
(43) Pub. Date: Jan. 22, 2015

(54) DOUBLE LINE IMAGING DEVICE

(71) Applicant: Gyrus ACMI, INC. (d.b.a. Olympus Surgical Technologies America), Southborough, MA (US)

(72) Inventors: Doron Adler, Haifa (IL); Shai Finkman, Haifa (IL); Adi Navve, Kfar Saba (IL)

(21) Appl. No.: 14/312,733

(22) Filed: Jun. 24, 2014

Related U.S. Application Data
(60) Provisional application No. 61/856,710, filed on Jul. 21, 2013.

(51) Int. Cl. A61B 1/05; A61B 1/00

(52) U.S. Cl. CPC A61B 1/05; A61B 1/0008; A61B 1/00183
USPC 600/407

(57) ABSTRACT

An imaging device, including a first longitudinal body which is configured to be inserted into a cavity, and a first linear imaging element having a first linear proximal end and a first linear distal end, which is rotatably connected at the first linear proximal end to the first longitudinal body. The imaging device further includes a second linear imaging element having a second linear proximal end and a second linear distal end, which is rotatably connected at the second linear proximal end to the first linear distal end. The device also has a second longitudinal body, slidably positioned with respect to the first longitudinal body and rotatably connected to the second linear distal end. There is a rotatable shaft that is connected to at least one of the first longitudinal body and the second longitudinal body.

### CROSS-REFERENCE TO RELATED APPLICATION

The present application claims the benefit of U.S. Provisional Patent Application 61/856,710, filed 21 Jul. 2013 and titled Double Line Imaging Device, which is incorporated herein by reference.

### FIELD OF THE INVENTION

The present invention relates generally to imaging, and specifically to endoscopic imaging.

### BACKGROUND OF THE INVENTION

U.S. Pat. No. 5,827,176 to Tanaka et al., whose disclosure is incorporated herein by reference, describes an endoscopic imaging system with a rotating line sensor. The line sensor is mounted on a shaft in the distal end of an endoscope so that the line sensor is separate from and parallel to the axis of the shaft. The distal end has a transparent casing and rotation of the shaft causes the line sensor to scan through the casing.

Japanese Patent JPH02-11117, to Sekiguchi Tadashi, whose disclosure is incorporated herein by reference, describes an electronic endoscope having a line sensor. In one embodiment the line sensor is rotatably mounted on a semi-disk shape base which in turn is mounted on a rotary base. The disclosure asserts that rotating the line sensor using this mechanism eliminates a restriction in the visual field.

U.S. Pat. No. 7,543,512 to Smith et al., whose disclosure is incorporated herein by reference, describes a probe for inspecting a bore. The probe includes a housing dimensioned for insertion into the bore, and a plurality of stabilization legs having a first end attached to the housing and a second end extendable from the housing. Sensors may be attached to the extendable ends and the disclosure claims that a video camera may be positioned to view an area sensed by the sensors.

Japanese Patent Application JP2009-297425, to Yamane Kenji, whose disclosure is incorporated herein by reference, describes an endoscope which is able to be inserted into a narrow opening. The endoscope distal portion has a cylindrical body which is able to incline and the disclosure claims that a lens in the inclined cylindrical body is able to acquire peripheral image information over a wide range.

Documents incorporated by reference in the present patent application are to be considered an integral part of the application except that to the extent that any terms are defined in these incorporated documents in a manner that conflicts with definitions made explicitly or implicitly in the present specification, only the definitions in the present specification should be considered.

### SUMMARY OF THE INVENTION

An embodiment of the present invention provides an imaging device, including:

a first longitudinal body which is configured to be inserted into a cavity;

a first linear imaging element having a first linear proximal end and a first linear distal end, rotatably connected at the first linear proximal end to the first longitudinal body;

a second linear imaging element having a second linear proximal end and a second linear distal end, rotatably connected at the second linear proximal end to the first linear distal end;

a second longitudinal body, slidably positioned with respect to the first longitudinal body and rotatably connected to the second linear distal end; and

a rotatable shaft connected to at least one of the first longitudinal body and the second longitudinal body.

The imaging device may also include:

a proximal hinge which rotatably connects the first linear proximal end to the first longitudinal body;

a central hinge which rotatably connects the second linear proximal end to the first linear distal end; and

a distal hinge which rotatably connects the second longitudinal body distal end to the second linear distal end.

In a disclosed embodiment respective axes of rotation of the proximal, central, and distal hinges are parallel. In a further disclosed embodiment the first longitudinal body includes a tube wherein in an un-deployed state of the imaging device a distance between the central hinge and the first and second linear imaging elements is less than a diameter of the tube. In a yet further disclosed embodiment the first longitudinal body includes a tube wherein in a deployed state of the imaging device a distance between the central hinge and the first and second linear imaging elements is greater than a diameter of the tube.

In an alternative embodiment the first longitudinal body includes a tube, wherein the first and second linear imaging elements are located at an opening of the tube proximal to a distal end of the tube.

In a further alternative embodiment the first longitudinal body includes a wire. In a yet further alternative embodiment the second longitudinal body includes a wire.

Typically, the first linear imaging element and the second linear imaging element are configured to acquire images of slices of the cavity on rotation of the rotatable shaft, and the imaging device includes a processor configured to stitch the images into a panoramic image of the cavity and to display the panoramic image.

There is further provided, according to an embodiment of the present invention embodiment of the present invention, a method, including:

configuring a first longitudinal body to be inserted into a cavity;

rotatably connecting a first linear imaging element, having a first linear proximal end and a first linear distal end, at the first linear proximal end to the first longitudinal body;

rotatably connecting a second linear imaging element, having a second linear proximal end and a second linear distal end, at the second linear proximal end to the first linear distal end;

slidably positioning a second longitudinal body with respect to the first longitudinal body;

rotatably connecting the second longitudinal body to the second linear distal end; and

connecting a rotatable shaft to at least one of the first longitudinal body and the second longitudinal body.

The present invention will be more fully understood from the following detailed description of the embodiments thereof, taken together with the drawings in which:

### BRIEF DESCRIPTION OF THE DRAWINGS

FIG. 1 is a schematic illustration of a double line imaging system, according to an embodiment of the present invention;

FIG. 2 is a schematic cross-sectional side view of an imaging device before it extends from the distal end of an insertion element retaining the device, when the imaging device is in an un-deployed configuration, according to an embodiment of the present invention;

FIG. 3 is a schematic cross-sectional side view of the imaging device in its un-deployed configuration after it has extended from the distal end, according to an embodiment of the present invention;

FIG. 4 is a schematic cross-sectional side view of the imaging device in a deployed configuration, according to an embodiment of the present invention;

FIG. 5 is a schematic perspective view of the imaging device in the deployed configuration, according to an embodiment of the present invention;

FIG. 6 is a schematic cross-sectional side view of an imaging device in a deployed configuration, according to an alternative embodiment of the present invention;

FIG. 7 is a schematic cross-sectional side view of an imaging device in a deployed configuration, according to a further alternative embodiment of the present invention; and

FIG. 8 is a flowchart of steps using the imaging device of FIGS. 2-5 to generate a panoramic image of a body cavity, according to an embodiment of the present invention.

### DETAILED DESCRIPTION OF EMBODIMENTS

### Overview

There is substantial clinical value in offering a physician a complete panoramic view of a lumen, such a view providing the ability to spot abnormalities with wide spread characteristics, ensure complete, gap free scanning of the lumen, and facilitate standardization and documentation of the results. Wide angle optics may provide a solution, but result in extremely distorted images, which are unacceptable in many fields including medical fields. Furthermore, in the medical field, there are serious physical limitations to an optical system that is intended to be deployed in an endoluminal cavity in a minimal invasive approach. Since such a system is to be delivered via an insertion element it must be small in its diameter.

Embodiments of the present invention provide an imaging device that may be delivered via an insertion element, which may typically comprise a catheter. The imaging device comprises two linear imaging elements. A first, proximal, of the elements is rotatably connected at its proximal end by a hinge to a first longitudinal body, herein assumed to comprise an imaging device tube. The imaging device tube has a diameter enabling it to slide within the insertion element. A second, distal, of the elements is rotatably connected at its distal end by another hinge to a second longitudinal body, herein assumed to comprise a backbone wire, which traverses the imaging device tube. The two linear imaging elements are rotatably connected by a third hinge to each other.

The imaging device has two configurations. In an un-deployed configuration the backbone wire is moved with respect to the imaging device tube so that the two linear elements, the tube, and the wire are all approximately parallel. In the un-deployed configuration the imaging device is approximately one-dimensional, and has dimensions enabling it to slide within the insertion element. In a deployed configuration the backbone wire is moved proximally with respect to the imaging device tube so that the two linear elements and a portion of the wire have a triangular shape, so that in the deployed configuration the imaging device is two-dimensional.

To image a cavity the imaging device is first introduced into the insertion element, and the imaging device in its un-deployed configuration is slid through the insertion element until it is within the cavity. The backbone wire is then moved proximally with respect to the imaging device tube, so placing the imaging device into its deployed configuration. In the deployed configuration the two linear imaging elements typically have a field of view of a slice of the cavity greater than 180°. By rotating the imaging device in its deployed configuration (around an axis defined by the imaging device tube) multiple slices of the cavity may be acquired, and the multiple slices may be stitched together to form a panoramic image of the cavity.

### DETAILED DESCRIPTION

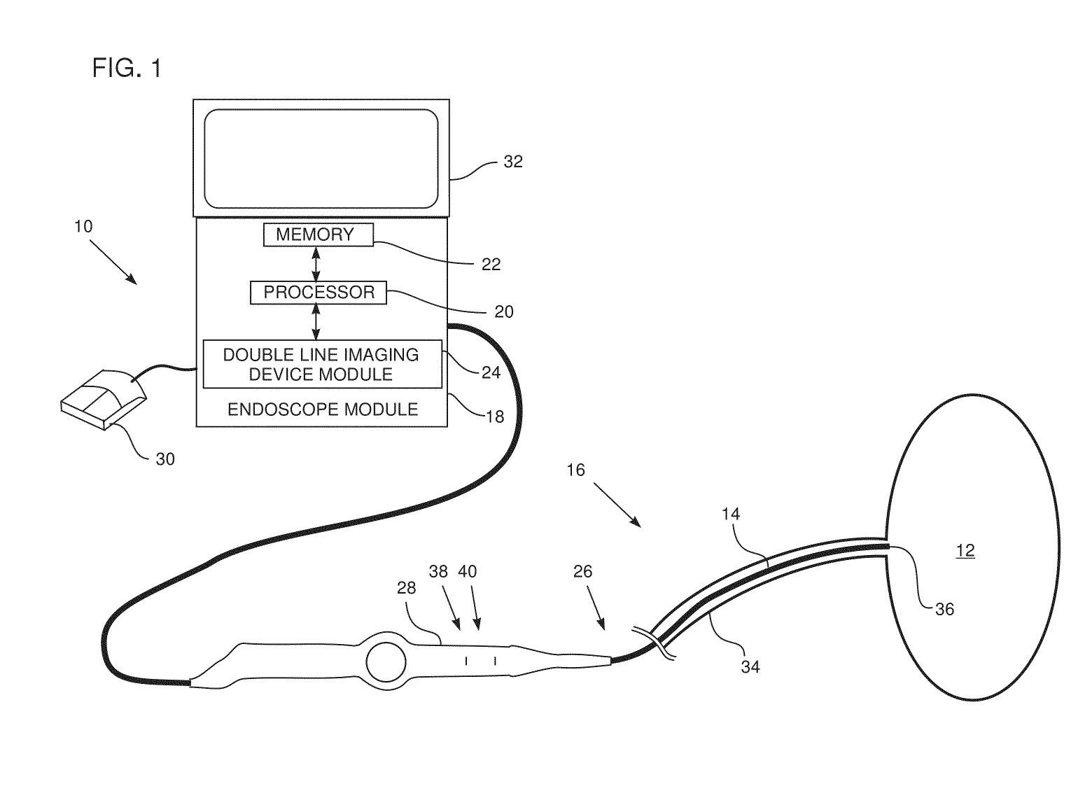

FIG. 1 is a schematic illustration of a double line imaging system 10, according to an embodiment of the present invention. System 10 may be used in an invasive medical procedure, typically a minimally invasive procedure, on a body cavity 12 of a human patient in order to image substantially the whole of the body cavity in a panoramic manner. By way of example, in the present description the body cavity is assumed to be the bladder of a patient, and body cavity 12 is also referred to herein as bladder 12. However, it will be understood that system 10 may be used to image substantially any human body cavity, such as the gastrointestinal organs, the bronchium, the chest, or a non-human cavity.

System 10 comprises an insertion element 14 which enables delivery of a double line imaging device 16 to bladder 12, so that the insertion element together with the imaging device acts as an endoscope. In an embodiment insertion element 14 comprises a catheter. Insertion element 14 is in the form of a tube which is able to traverse a lumen of a patient's body, and insertion element 14 is also referred to herein as insertion element tube 14, or just as tube 14. Imaging device 16, which is installed within tube 14 and which is operative at a distal end of the tube, is controlled by an endoscope module 18 having a processor 20 communicating with a memory 22. Endoscope module 18 also comprises a double line imaging device module 24, whose functions are described below, and which may be implemented in software, hardware, or a combination of software and hardware. Insertion element 14 is connected at its proximal end 26 to a handle 28 which enables an operator, herein assumed to be a physician, of system 10 to insert the insertion element into the bladder as well as to manipulate imaging device 16 so as to acquire images of the bladder. A motor 38 may be installed in handle 28, and a rotatable shaft 40 of the motor may be connected to imaging device 16. The motor and its shaft, described in more detail below, enable manipulation of imaging device 16.

In some embodiments of the present invention, rather than manual manipulation of imaging device 16 using handle 28, the imaging device is manipulated automatically, such as by scanning, so as to acquire its images. U. S. Patent Application 2009/0177034, which is incorporated herein by reference, describes a method for automatic scanning of an endoscope, and the method may be adapted, mutatis mutandis, for embodiments of the present invention wherein automatic scanning is used. For simplicity, except where otherwise stated, in the following description manual imaging device manipulation using handle 28 is assumed, and those having ordinary skill in the art will be able to adapt the description for the case of automatic imaging device manipulation. The operator is able to provide input to module 18 via controls 30, which typically comprise at least one of a keyboard, a pointing device, or a touch screen. Alternatively or additionally, at least some of controls may be incorporated in handle 28. For simplicity, controls 30 are herein assumed to comprise a mouse.

The processor uses software, typically stored in memory 22, to control system 10. Results of the actions performed by processor 20 may be presented on a screen 32 to the operator of system 10, the screen typically displaying a panoramic image of bladder 12 that is generated by system 10. The software for operating system 10 may be downloaded to processor 20 in electronic form, over a network, for example, or it may, alternatively or additionally, be provided and/or stored on non-transitory tangible media, such as magnetic, optical, or electronic memory.

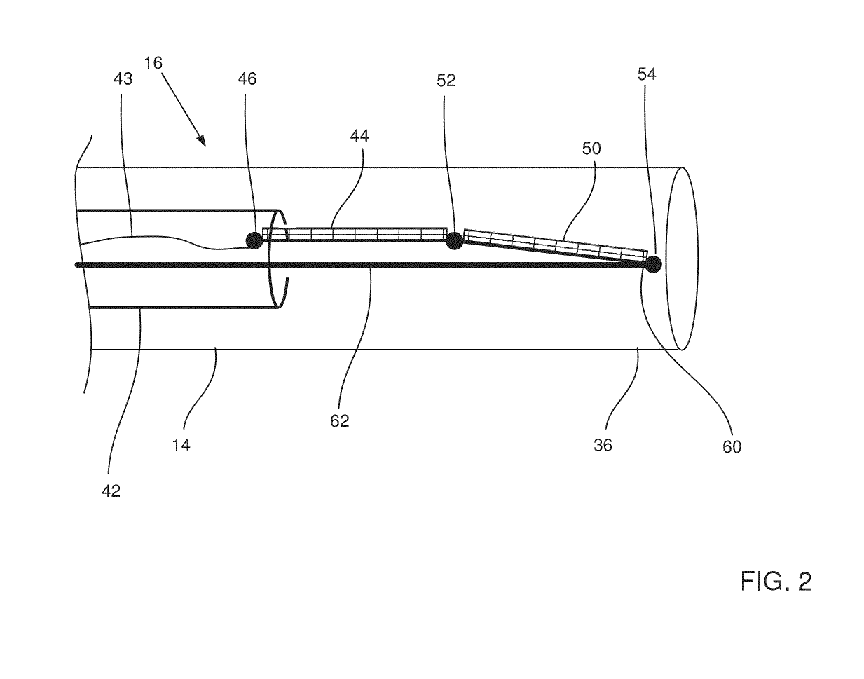

To operate system 10, the physician inserts insertion element 14 through a urethra 34 until a distal end 36 of the insertion element enters the bladder. FIG. 2 is a schematic cross-sectional side view of imaging device 16 before it extends from distal end 36, and when the imaging device is in an un-deployed configuration, according to an embodiment of the present invention. Distal end 36 is at the termination of insertion element tube 14, and imaging device 16 comprises a first longitudinal body 42, which is dimensioned so that it is able to traverse tube 14, and is also able to exit from distal end 36. In a disclosed embodiment first longitudinal body 42 comprises a tube, and in the following description of the disclosed embodiment, body 42 may also be referred to as an imaging device external tube, or just as a tube. While the figure illustrates imaging device 16 as being at distal end 36, it will be understood that since the imaging device can slide within insertion element tube 14, imaging device 16 may be located substantially anywhere within the insertion element tube, and may even be configured to be removable from the proximal end of the insertion element tube.

In imaging device 16 a first linear imaging element 44 is connected, at its proximal end, by a proximal hinge 46 to the distal end of tube 42. A second linear imaging element 50, typically similar in dimensions and properties to the first linear imaging element, is connected at its proximal end by a central hinge 52 to the distal end of the first linear imaging element. A distal hinge 54 connects the distal end of the second linear element to a distal end 60 of a second longitudinal body 62, which is configured to slide within imaging device external tube 42.

A cable 43, typically an electrically conductive cable, is located in proximity to first longitudinal body and is configured to transfer signals between first and second linear imaging elements 44 and 50 and module 18. If first longitudinal body 42 comprises a tube, cable 43 may be located within the tube, as illustrated in FIG. 2. Cable 43 may be connected directly to both elements 44 and 50. Alternatively cable 43 may be connected to only one of elements 44 or 50, in which case hinge 42 may be configured, typically by the hinge electrically coupling the two elements, to transfer signals between the cable and the element not connected to the cable.

In the disclosed embodiment body 62 comprises a wire that acts as a “backbone” of device 16, so that body 62 is also referred to herein as a backbone wire. In order to be able to traverse tube 14, wire 62 may be locked to imaging device external tube 42, typically at their proximal ends using controls in handle 28, so as to maintain the un-deployed configuration of the imaging device illustrated in FIG. 2. In its un-deployed configuration, linear imaging elements 44 and 50 are approximately in line with each other, in an approximately one-dimensional configuration and are approximately parallel to backbone wire 62. In the un-deployed configuration a largest distance d between the elements and the backbone wire is less than the diameter of imaging device external tube 42.

Hinges 46, 52, and 54 are all generally similar in construction, and all three hinges are configured to rotate about respective axes which are parallel to each other. In the present description, for simplicity, the rotation axes for hinges 46, 52, and 54 are assumed to be orthogonal to the plane of the paper.

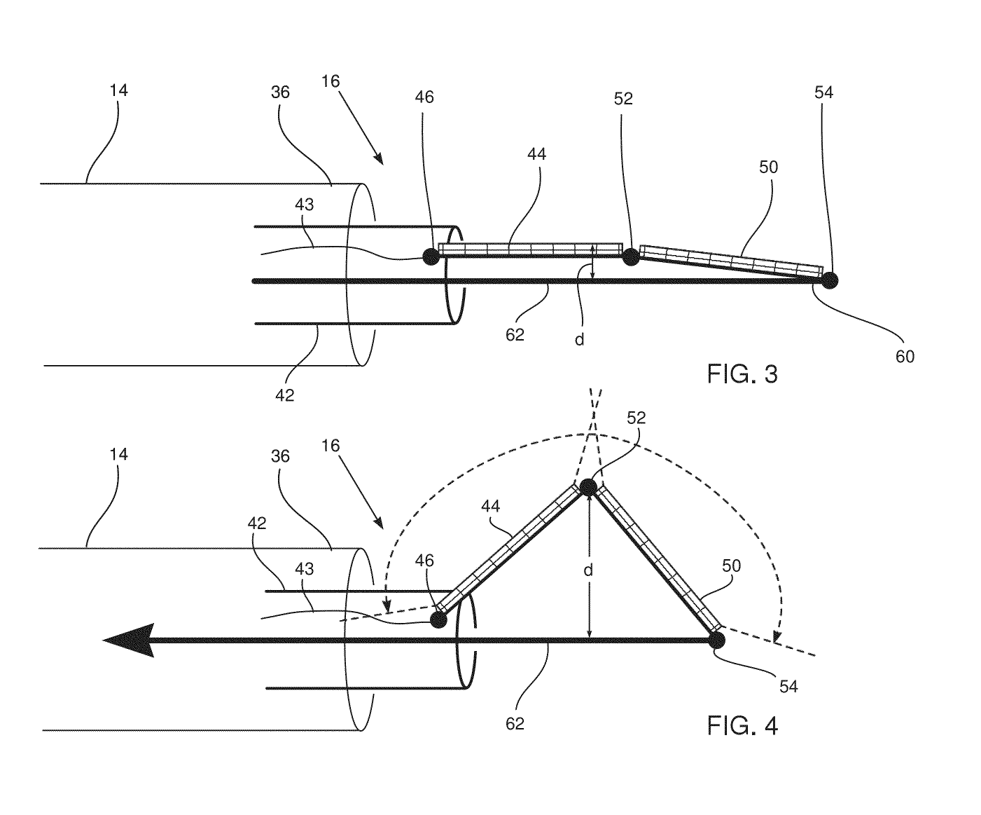

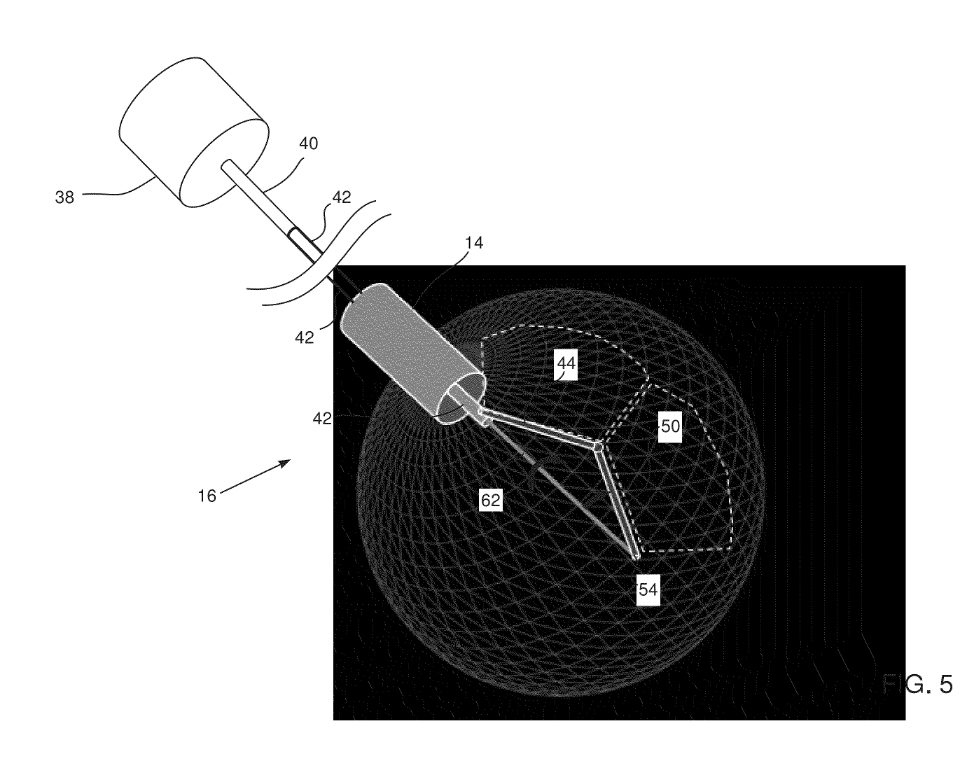

FIG. 3 is a schematic cross-sectional side view of imaging device 16 in its un-deployed configuration after it has extended from distal end 36, according to an embodiment of the present invention. FIG. 4 is a schematic cross-sectional side view of the imaging device in a deployed configuration, and FIG. 5 is a schematic perspective view of the imaging device in the deployed configuration, according to an embodiment of the present invention.

In order to operate imaging device 16, the device, in its un-deployed configuration, is slid from insertion element tube 14 so that it extends beyond distal end 36 of the insertion element tube, into bladder 12, as illustrated by FIG. 3.

Once positioned beyond distal end 36, the imaging device may transfer to its deployed configuration, as illustrated in FIGS. 4 and 5. In order to transfer from the un-deployed configuration to the deployed configuration, backbone wire 62 and tube 42 are unlocked, so that they are able to move with respect to each other. The deployed configuration is then attained by moving backbone wire 62 in a proximal direction, as illustrated by the arrow head in FIG. 4, while keeping tube 42 substantially fixed. Once in its deployed configuration, wire 62 and tube 42 may be locked in position, to maintain the imaging device in its deployed configuration.

In attaining the deployed configuration, wire 62 moves proximally relative to tube 42, causing all three hinges 46, 52, and 54 to rotate. In FIG. 5 the single headed arrow illustrates rotation of hinge 54. The hinge rotation causes the largest distance d between the linear imaging elements and the backbone wire to increase from its un-deployed value and to be greater than the diameter of insertion element tube 14. The largest distance d corresponds to the distance between central hinge 52 and backbone wire 62. In the deployed configuration the imaging device is in a two-dimensional configuration, wherein elements 44, 50, and a portion of wire 62 approximately form a triangle with a non-zero height d.

Once imaging device 16 is in its deployed configuration, module 24 (FIG. 1) may activate the linear imaging elements of the imaging device, in order to acquire an image. A field of view of each of the linear elements is indicated schematically in FIGS. 4 and 5 by straight broken lines. While each of the linear elements of imaging device 16 may have a respective field of view less than 180°, an overall field of view of the imaging device is greater than 180°, as is indicated schematically in FIG. 4 by the double-headed curved broken line. Consequently, an image formed by imaging device 16 in its deployed configuration corresponds to a slice of the wall of cavity 12.

To form a panoramic image of the wall of cavity 12, tube 42 is rotated about its axis, while imaging device is in its deployed configuration, so as to sequentially scan the wall. To perform the rotation, shaft 40 of motor 38 is connected to tube 42, and the motor is activated. The rotation is illustrated by the double-headed arrow in FIG. 5. The scanning, i.e., the axial rotation, may be manual or automatic, as described above. During the scanning module 24 acquires multiple sequential images of slices of the wall of cavity 12, and the module stitches the images together using any convenient stitching method known in the art, such as by matching features of the wall that are common to different image slices.

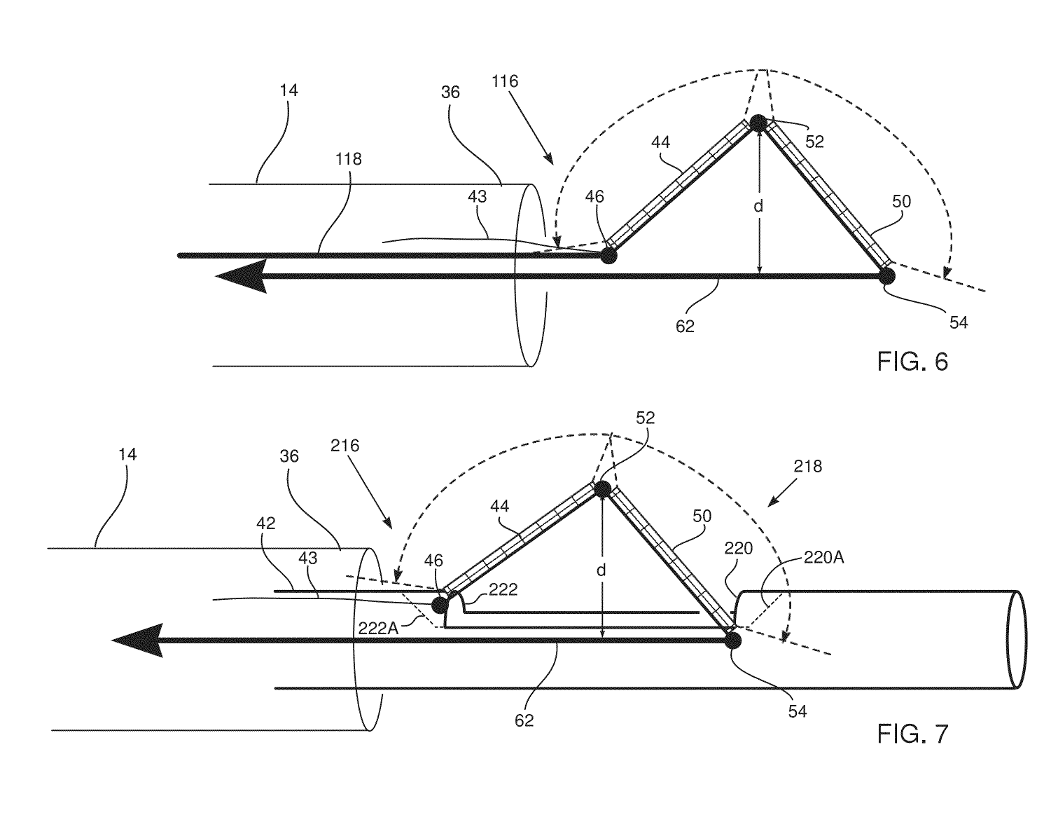

FIG. 6 is a schematic cross-sectional side view of an imaging device 116 in a deployed configuration, according to an alternative embodiment of the present invention. Apart from the differences described below, the operation of imaging device 116 is generally similar to that of imaging device 16 (FIGS. 1-5), and elements indicated by the same reference numerals in both imaging device 116 and 16 are generally similar in construction and in operation.

In contrast to imaging device 16, wherein the first longitudinal body comprises a tube, in imaging device 116 the first longitudinal body comprises a wire 118, typically generally similar to backbone wire 62. Imaging device 116 is deployed and un-deployed in a generally similar manner to that described for device 16, by moving backbone wire 62 longitudinally with respect to wire 118. In its deployed state, imaging device 116 may be rotated by rotating at least one of, and typically both, wires 118 and wire 62.

FIG. 7 is a schematic cross-sectional side view of an imaging device 216 in a deployed configuration, according to a further alternative embodiment of the present invention. Apart from the differences described below, the operation of imaging device 216 is generally similar to that of imaging device 16 (FIGS. 1-5), and elements indicated by the same reference numerals in both imaging device 216 and 16 are generally similar in construction and in operation.

In contrast to imaging device 16, wherein first and second linear imaging elements 44 and 50 extend beyond a distal end of tube 42, in imaging device 216 first and second linear imaging elements 44 and 50 are held in an opening 218 of tube 42. Opening 218 is located in a region of tube 42 that is proximal to the distal end of the tube. In its un-deployed configuration, the linear imaging elements of device 216 are retained within the opening. In its deployed configuration, as shown in FIG. 7, the linear imaging elements extend from the opening.

In some embodiments, ends 220 and/or 222 of opening 218 may be beveled, as indicated schematically by lines 220A and 222A. The beveling prevents vignetting of the fields of view of the linear imaging elements.

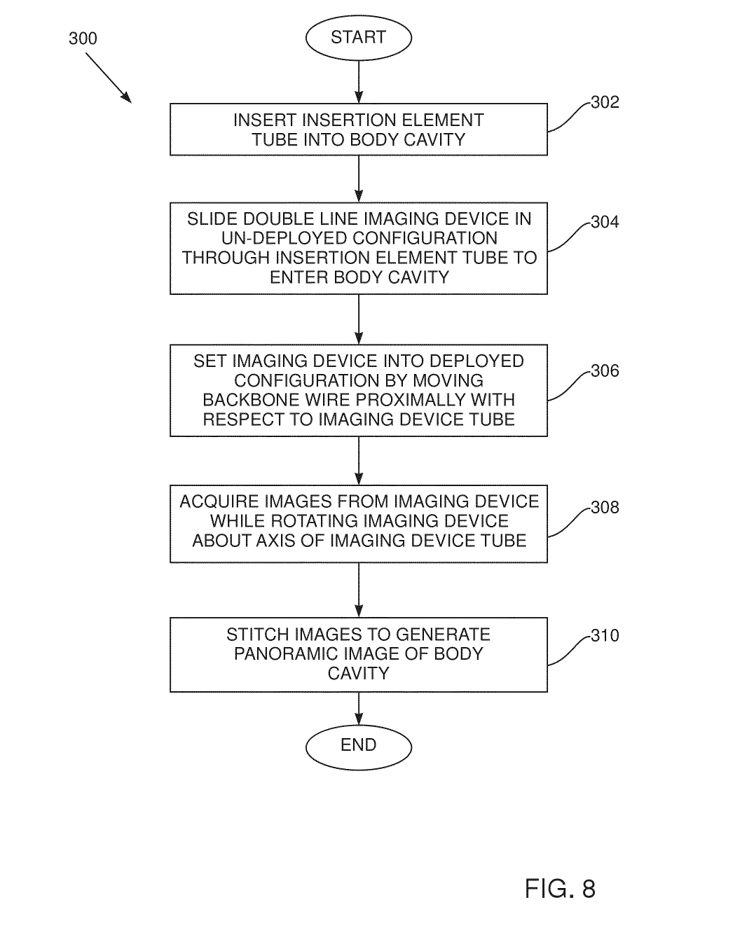

FIG. 8 is a flowchart 300 of steps using imaging device 16 to generate a panoramic image of a body cavity, according to an embodiment of the present invention embodiment of the present invention. Those having ordinary skill in the art will be able to adapt the following description, mutatis mutandis, for imaging devices 116 and 216.

In an initial step 302, insertion element tube 14 is inserted into cavity 12, typically as described above with respect to FIG. 1.

In a cavity entry step 304, double line imaging device 16 is slid, in its un-deployed configuration, through insertion element tube 14 until the imaging device enters cavity 12, generally as illustrated above in FIG. 3.

In an imaging device deployment step 306, the imaging device is transferred into its deployed configuration by moving backbone wire 62 in a proximal direction with respect to imaging device tube 42, as described above with reference to FIGS. 3, 4, and 5.

In an image acquisition step 308, the imaging device is rotated about the axis of imaging device tube 42. As the imaging device is rotated into different sequential orientations, module 24 acquires respective image slices of the wall of cavity 12, as described above.

In an image generation step 310, module 24 stitches the image slices together to form a panoramic image of the wall of cavity 12, and displays the stitched image on screen 32.

While the description above has assumed generally that double line imaging system 10 generates an image of a body cavity, it will be appreciated that the system may be used to generate an image of any cavity, human or non-human. The system may advantageously be used where entry to the cavity is restricted, for example, if the cavity comprises the bladder, and entry is via the urethra.

While the description above has assumed that imaging system 10 comprises two linear imaging elements rotatably connected together, the scope of the present invention includes more than two linear elements rotatably connected together. Thus, embodiments of the present invention include three, four, or more linear imaging elements rotatably connected to each other so as to form a hinged linear chain. For such embodiments the proximal end of the first linear imaging element in the chain is rotatably connected to a first longitudinal body, and the distal end of the last linear element in the chain is rotatably connected to a second longitudinal body. The first and second longitudinal bodies of such embodiments may respectively correspond to the first and second longitudinal bodies of devices 16, 116, or 216.

It will be understood that embodiments having more than two linear imaging elements are able to form a panoramic image, by methods substantially similar to those described above for embodiments having two linear imaging elements.

It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

We claim:

1. An imaging device, comprising:
a first longitudinal body which is configured to be inserted into a cavity;
a first linear imaging element having a first linear proximal end and a first linear distal end, rotatably connected at the first linear proximal end to the first longitudinal body;
a second linear imaging element having a second linear proximal end and a second linear distal end, rotatably connected at the second linear proximal end to the first linear distal end;
a second longitudinal body, slidably positioned with respect to the first longitudinal body and rotatably connected to the second linear distal end; and
a rotatable shaft connected to at least one of the first longitudinal body and the second longitudinal body.

2. The imaging device according to claim 1, and comprising:
a proximal hinge which rotatably connects the first linear proximal end to the first longitudinal body;
a central hinge which rotatably connects the second linear proximal end to the first linear distal end; and
a distal hinge which rotatably connects the second longitudinal body distal end to the second linear distal end.

3. The imaging device according to claim 2, wherein respective axes of rotation of the proximal, central, and distal hinges are parallel.

4. The imaging device according to claim 2, wherein the first longitudinal body comprises a tube and wherein in an un-deployed state of the imaging device a distance between the central hinge and the first and second linear imaging elements is less than a diameter of the tube.

5. The imaging device according to claim 2, wherein the first longitudinal body comprises a tube and wherein in a deployed state of the imaging device a distance between the central hinge and the first and second linear imaging elements is greater than a diameter of the tube.

6. The imaging device according to claim 1, wherein the first longitudinal body comprises a tube, and wherein the first and second linear imaging elements are located at an opening of the tube proximal to a distal end of the tube.

7. The imaging device according to claim 1, wherein the first longitudinal body comprises a wire.

8. The imaging device according to claim 1, wherein the second longitudinal body comprises a wire.

9. The imaging device according to claim 1, wherein the first linear imaging element and the second linear imaging element are configured to acquire images of slices of the cavity on rotation of the rotatable shaft, and comprising a processor configured to stitch the images into a panoramic image of the cavity and to display the panoramic image.

10. A method, comprising:
configuring a first longitudinal body to be inserted into a cavity;
rotatably connecting a first linear imaging element, having a first linear proximal end and a first linear distal end, at the first linear proximal end to the first longitudinal body;
rotatably connecting a second linear imaging element, having a second linear proximal end and a second linear distal end, at the second linear proximal end to the first linear distal end;
slidably positioning a second longitudinal body with respect to the first longitudinal body;
rotatably connecting the second longitudinal body to the second linear distal end; and
connecting a rotatable shaft to at least one of the first longitudinal body and the second longitudinal body.

11. An imaging device, comprising:
a first longitudinal body which is configured to be inserted into a cavity;
three or more linear imaging elements rotatably connected together so as to form a linear chain, the chain having a chain proximal end and a chain distal end, the chain being rotatably connected at the chain proximal end to the first longitudinal body;
a second longitudinal body, slidably positioned with respect to the first longitudinal body and rotatably connected to the chain distal end; and
a rotatable shaft connected to at least one of the first longitudinal body and the second longitudinal body.
